(19) United States
(12) Patent Application Publication
(10) Pub. No.: US 2014/0378781 A1
(43) Pub. Date: Dec. 25, 2014

(54) METHODS AND SYSTEMS FOR ACQUIRING DIAGNOSTIC INFORMATION

(71) Applicant: QARDIO, INC., Walnut, CA (US)

(72) Inventors: Marco PELUSO, London (GB); Rosario IANNELLA, Amsterdam (NL)

(21) Appl. No.: 14/312,830

(22) Filed: Jun. 24, 2014

Related U.S. Application Data
(60) Provisional application No. 61/838,953, filed on Jun. 25, 2013.

(51) Int. Cl. A61M 21/02; A61B 5/145; A61B 5/00; A61B 5/0402; A61B 5/01; A61B 5/021; A61B 5/024

(52) U.S. Cl. CPC A61M 21/02; A61B 5/021; A61B 5/14532; A61B 5/02405; A61B 5/0402; A61B 5/14542; A61B 5/01; A61B 5/0015
USPC 600/301

(57) ABSTRACT

Methods are described to enable a patient to conveniently test a biological parameter remote from a clinic setting (e.g., at home or work) which provides a way to reduce stress associated with the process. The devices and methods are also adaptable to provide resulting information via a communication network to a remote location and/or a third party (health care provider, health buddy, etc.).

### CROSS-REFERENCE

This application claims the benefit of U.S. Provisional Application No. 61/838,953, filed Jun. 25, 2013, entitled METHODS AND SYSTEMS FOR ACQUIRING DIAGNOSTIC INFORMATION by Marco Peluso, et al., which application is incorporated herein by reference.

### BACKGROUND OF THE INVENTION

The number of healthcare practitioners available to provide healthcare to a growing and aging population is under strain. Moreover, people are increasingly moving to a model where they take a more active role in their ongoing healthcare monitoring and maintenance. This has led to a rise in the mobile health market, which is facilitated by smart devices (phones, tablets, etc.) that are portable and have increasing computing power as well as by the availability of medical information on the Internet. Moving into the 21^{st} century, most would agree that consumer self-awareness and self-control monitoring is here. This has led to an increase in the market for devices and test kits for at-home diagnosing: which increased 10% in 2012 and 35% between 2010-2011.

Regular measurement of blood pressure is an important part of antihypertensive or hypotensive therapy, as well as the control of certain side-effects caused by certain therapies for various kinds of applications. According to the guidelines for antihypertensive therapies of the World Health Organization (“WHO”), the degrees of hypertension are classified in accordance with blood pressure values measured every 5 mmHg, and therapeutic methods suitable for the individual degrees are recommended. Therefore, whether appropriate therapies can be performed depends on the measured blood pressure values. Also, as the aging population advances, demands for high accuracy and high reliability of blood pressure measurement are on the rise in order to prevent circulatory organ diseases and metabolic syndrome which is impacted by hypertension.

Patients diagnosed with high blood pressure are advised to engage in a regime of home monitoring—typically once or twice a day—in order to quickly identify changes in blood pressure control. A number of devices are available for home blood pressure measurement. Digital blood pressure devices are considered easy to use: they automatically calculate the pulse and display the systolic and diastolic pressures. However, a simple upper arm blood pressure cuff with a stethoscope is often considered by doctors the most reliable way to measure blood pressure. Omron Healthcare has a variety of products. Additionally Omron Healthcare holds several patents for blood pressure measurement devices including, for example, U.S. Pat. No. 4,776,344 A issued Oct. 11, 1988, for Electronic Blood Pressure Measuring Device; U.S. Pat. No. 7,794,405 B2 issued Sep. 14, 2010, for Cuff for Blood Pressure Monitor, and Blood Pressure Monitor Having the Same; U.S. Pub US 2013/0138000 A1 published May 30, 2013, for Blood Pressure Measurement Device. Heart rate monitors are disclosed in, for example, U.S. Pat. No. 4,625,733 A issued Dec. 2, 1986 for Procedure and Means for Telemetric Measuring of Heartbeat and ECG Signal, Using a Magnetic Proximity Field by Saynajakangas; and U.S. Pat. No. 5,464,021 A issued Nov. 7, 1995 for Telemetric Transmitter Unit by Birnbaum.

Technology has come a long way to enable patients to monitor their blood pressure in home. However, research suggests that up to one-third of people who were thought to have treatment-resistant may actually have “white coat hypertension”—which results in blood pressure, heart rate, or blood sugar spiking in the doctor's office. Patients also see increases in blood pressure, heart rate, and blood sugar when stressed.

What is needed is a method for patients to test a biological parameter remote from a clinic setting (e.g., at home or work) which also provides a way to reduce stress associated with the process thereby providing a more accurate reading of the parameter.

### SUMMARY OF THE INVENTION

Methods are described to enable a patient to conveniently test a biological parameter remote from a clinic setting (e.g., at home or work) which provides a way to reduce stress associated with the process. The devices and methods are also adaptable to provide resulting information via a communication network to a remote location and/or a third party (health care provider, health buddy, etc.).

Other diagnostics that can be monitored outside the clinical setting using the devices, methods and systems disclose include, for example, blood sugar (glucose), heart rate, respiratory rate, electrocardiogram, blood oxygen, body temperature, skin galvanic response, weight, etc.

An aspect of the disclosure is directed to an electronic system for acquiring medical information. Systems comprise: a first biological parameter detection device configurable to acquire a biological parameter; and an electronic device having an electronic device memory, at least one of a display for generating a visual output and a speaker for generating an audio output, and a communicator configurable to establish a communication link with the first biological parameter detection device, wherein the electronic device is configurable to deliver at least one of a first visual and a first audio output in response to a start command. The start command can be generated on the biological parameter detection device or the electronic device in communication with the biological parameter detection device. In at least some configurations, the at least one of the display and/or speaker is pre-selected by a user. In other configurations, the at least one of the display and/or speaker output is automatically selected, semi-automatically selected, or selected by a caretaker. The first biological parameter detection device can be selected from a group comprising: blood pressure monitor, glucose monitor, heart rate monitor, electrocardiogram (“ECG” or “EKG”) monitor, respiratory monitor, blood oxygen monitor, body temperature monitor, and skin galvanic response monitor. Additionally, the first biological parameter detection device is configurable to provide the acquired biological parameter to a third party device. In some configurations, the system further comprises a second biological parameter detection device configurable to acquire a second biological parameter wherein the electronic device is configurable to generate at least one of a second visual output and a second audio output and further wherein the electronic device is configurable to deliver the second output in response to activation of the second biological parameter detection device. The second biological parameter detection device is also selected from a group comprising: blood pressure monitor, glucose monitor, heart rate monitor, ECG monitor, respiratory monitor, blood oxygen monitor, body temperature monitor, and skin galvanic response monitor. The second biological parameter detection device is typically a different device than the first biological parameter detection device and provides a second output is different than the first output. A playback of the first visual output and/or the first audio output can be synchronized to the biological measurement.

Another aspect of the disclosure is directed to a method for acquiring a biological parameter. The method comprises: activating a first biological parameter detection device configured to acquire a first biological parameter; activating an output of an electronic device having a device memory, at least one of a display for generating a visual output and a speaker for generating an audio output, and a communicator configurable to establish a communication link with the first biological parameter detection device; measuring a biological parameter with the first biological parameter detection device; determining an output associated with the first biological parameter detection device; and delivering the output associated with the first biological parameter detection device on the electronic device concurrently with the step of measuring. The method can further comprise pre-selecting the output of the electronic device. Pre-selection can be made by either the user or another party (such as a caretaker or a healthcare provider). The first biological parameter detection device can be selected from the group comprising: blood pressure monitor, glucose monitor, heart rate monitor, ECG monitor, respiratory monitor, blood oxygen monitor, body temperature monitor, and skin galvanic response monitor. The method can further comprise: activating a second biological parameter detection device configured to acquire a second biological parameter; determining an output associated with the second biological parameter detection device; and delivering an output associated with the second biological parameter detection device on the electronic device concurrently with the step of measuring. Additionally, the second biological parameter detection device can be selected from the group comprising: blood pressure monitor, glucose monitor, heart rate monitor, ECG monitor, respiratory monitor, blood oxygen monitor, body temperature monitor, and skin galvanic response monitor. The output associated with the first biological parameter detection device is different than the output associated with the second biological parameter detection device. An additional aspect can include providing the acquired biological parameter to a third party.

Still another aspect of the disclosure is directed to a machine readable medium containing machine readable instructions that, when executed by a computing device, cause the computing device to perform a method, the method comprising: activating a first biological parameter detection device configured to acquire a first biological parameter; activating an output of an electronic device having a device memory, at least one of a speaker and a display, and a communicator configurable to establish a communication link with the first biological parameter detection device; measuring a biological parameter with the first biological parameter detection device; determining an output associated with the first biological parameter detection device; and delivering the output associated with the first biological parameter detection device on the electronic device concurrently with the step of measuring. As previously discussed, the method can further comprise pre-selecting an output. In some aspects, the method can comprise selecting the first biological parameter detection device from a group comprising: blood pressure monitor, glucose monitor, heart rate monitor, ECG monitor, respiratory monitor, blood oxygen monitor, body temperature monitor, and skin galvanic response monitor. Still other configurations include activating a second biological parameter detection device configured to acquire a second biological parameter; determining an output associated with the second biological parameter detection device; and delivering an output associated with the second biological parameter detection device on the electronic device concurrently with the step of measuring. The second biological parameter detection device can also be selected from a group comprising: blood pressure monitor, glucose monitor, heart rate monitor, ECG monitor, respiratory monitor, blood oxygen monitor, body temperature monitor, and skin galvanic response monitor. Additionally, the acquired one or more biological parameters can be provided to a third party.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is flow diagram illustrating how a system is configured according to the disclosure;

FIG. 2 is a flow diagram illustrating how a system operates according to the disclosure; and

FIGS. 3A-B illustrates a communication network through which the devices disclosed can communicate data.

### DETAILED DESCRIPTION OF THE INVENTION

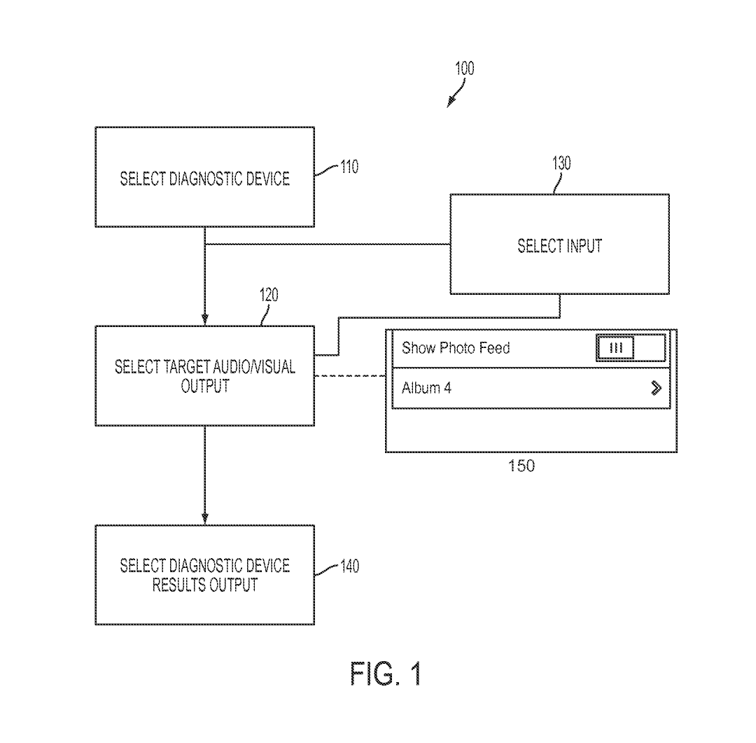

FIG. 1 is a flow diagram illustrating a process 100 for setting up a system according to the disclosure. During an initial set-up process, the user can select one or more diagnostic devices 110 which will be used as part of the system to acquire one or more target biological parameters. Parameters include, for example, blood pressure, blood sugar (glucose), heart rate, respiratory rate, electrocardiogram (ECG), blood oxygen, body temperature, skin galvanic response. A target audio and/or visual output 120 is then selected which can be associated with one or more diagnostic devices. The audio output includes, for example, a music of voice track which is played and is perceived via a speaker or headset. The visual output is, for example, a picture or video which is perceived via a screen. Alternatively, each diagnostic device can be associated with its own audio and/or video input 130, as shown by the screen shot insert 150 from 120. Moreover, each diagnostic device can be associated with more than one of each audio or visual output.

Thereafter, the user can select one or more output locations for the diagnostic measurement 140. Output locations can include an audio and/or visual indicator which will be activated (e.g., via a computing device via which the diagnostic device operates), or can transmit the sensed information to another party. Another party can be a caregiver, a physician, a healthcare provider, or any other person designated. The second party can be determined by the user or by a person setting up the device for the user.

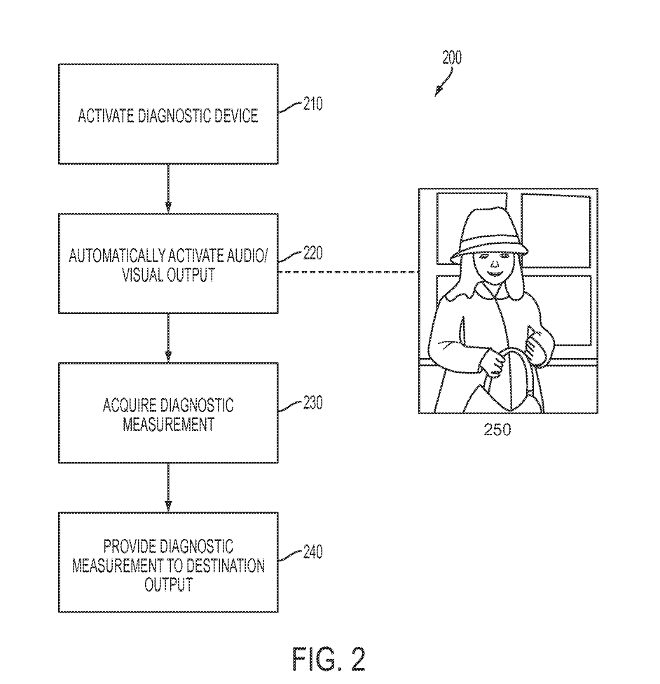

FIG. 2 is a flow diagram 200 illustrating a process for operating a diagnostic device according to the system. The user activates a diagnostic device 210. Thereafter the system automatically or semi-automatically activates an audio and/or video output 220 in communication with the diagnostic device, for example a photo 250 depicted in FIG.2 and the audio/video may or may not synchronized to the diagnostic measurement. Upon activation of the audio and/or video output, the diagnostic device begins acquiring a diagnostic measurement 230 of one or more target parameters (e.g., blood pressure, heart rate, blood sugar). During the acquiring process, the audio and/or visual output continues to be delivered to the user. Once the target diagnostic measurement is acquired, the results are provided to the destination output 240 identified by the user during the set-up process.

The automatically or semi-automatically activated audio and/or visual output can be any audio or visual output that is selected. Selection can be made by the user, for example, to induce a feeling of calm or happiness in the user. Thus, counteracting any “white coat” syndrome that might increase a reading taken by the device.

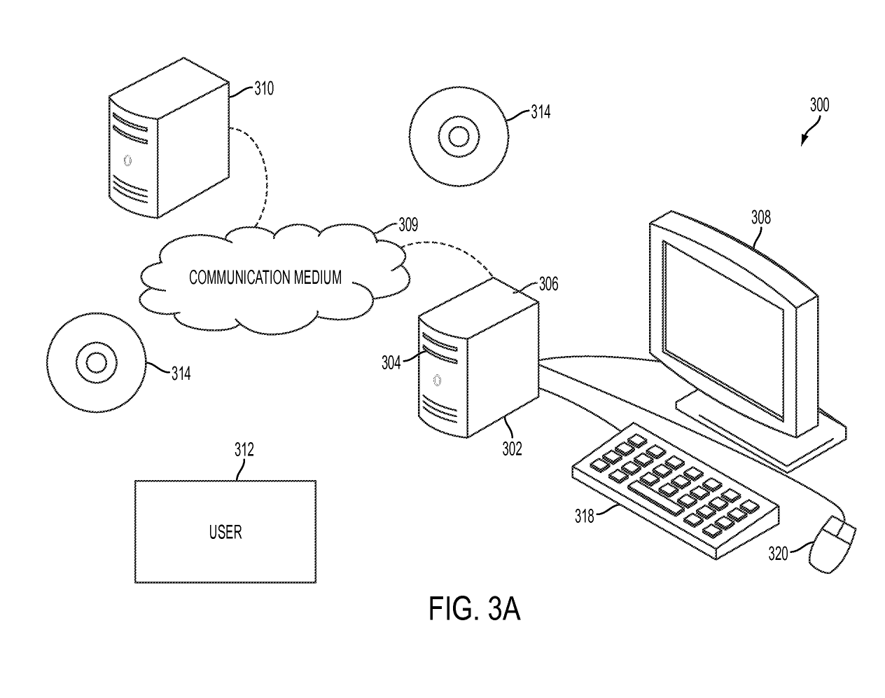

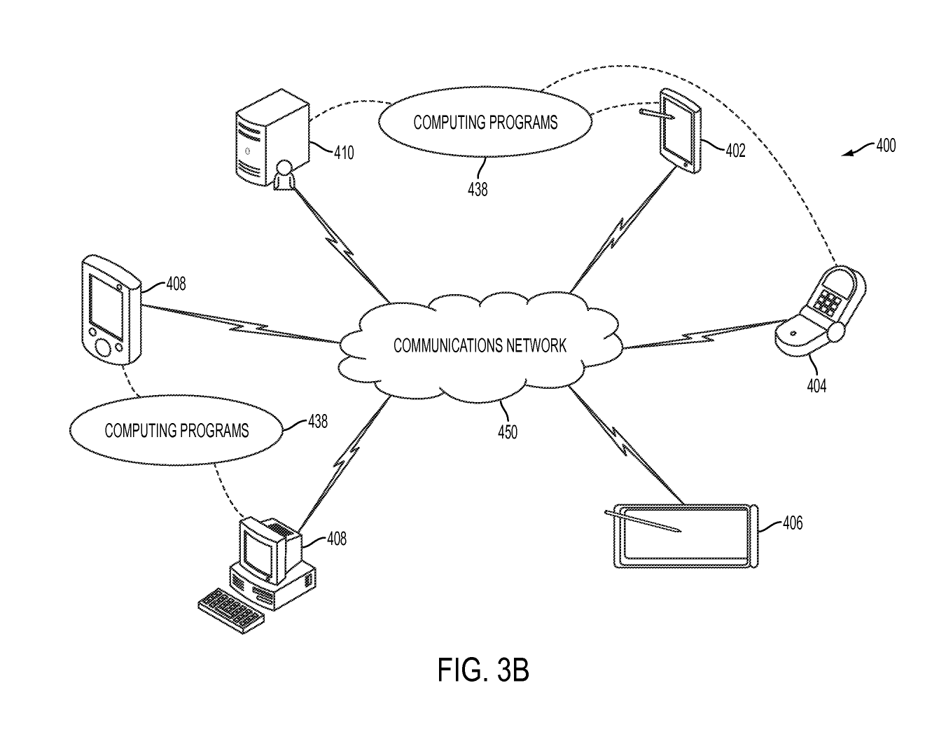

FIG. 3A is a block diagram showing a representative example logic device through which the display from the monitor can be achieved. A computer system (or digital device) 300, which may be understood as a logic apparatus adapted and configured to read instructions from media 314 and/or network port 306, is connectable to a server 310, and has a fixed media 316. The computer system 300 can also be connected to the Internet or an intranet. The system includes central processing unit (CPU) 302, disk drives 304, optional input devices, illustrated as keyboard 318 and/or mouse 320 and optional monitor 308. Data communication can be achieved through, for example, communication medium 309 to a server 310 at a local or a remote location. The communication medium 309 can include any suitable means of transmitting and/or receiving data. For example, the communication medium can be a network connection, a wireless connection or an internet connection. It is envisioned that data relating to the present invention can be transmitted over such networks or connections. The computer system can be adapted to communicate with a participant and/or a device used by a participant. The computer system is adaptable to communicate with other computers over the Internet, or with computers via a server.

As shown in FIG. 3B, the computing system 400 can be configured such that it is capable of executing a variety of computing applications 438, including computing applications, a computing applet, a computing program, or other instructions for operating on computing system 400 to perform at least one function, operation, and/or procedure. Computing system 400 is controllable by computer readable storage media for tangibly storing computer readable instructions, which may be in the form of software. The computer readable storage media adapted to tangibly store computer readable instructions can contain instructions for computing system 400 for storing and accessing the computer readable storage media to read the instructions stored thereon themselves. Such software may be executed within CPU 402 to cause the computing system 400 to perform desired functions. In many known computer servers, workstations and personal computers CPU 402 is implemented by micro-electronic chips CPUs called microprocessors. Optionally, a co-processor, distinct from the main CPU 402, can be provided that performs additional functions or assists the CPU 402. The CPU 402 may be connected to co-processor through an interconnect. One common type of coprocessor is the floating-point coprocessor, also called a numeric or math coprocessor, which is designed to perform numeric calculations faster and better than the general-purpose CPU 402.

As will be appreciated by those skilled in the art, a computer readable medium stores computer data, which data can include computer program code that is executable by a computer, in machine readable form. By way of example, and not limitation, a computer readable medium may comprise computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable storage media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other physical or material medium which can be used to tangibly store the desired information or data or instructions and which can be accessed by a computer or processor.

Some embodiments may be implemented in one or a combination of hardware, firmware and software. Embodiments may also be implemented as instructions stored on a non-transitory computer-readable storage medium, which may be read and executed by at least one processor to perform the operations described herein. A non-transitory computer-readable storage medium may include any mechanism for storing information in a form readable by a machine (e.g., a computer). For example, a non-transitory computer-readable storage medium may include read-only memory (ROM), random-access memory (RAM), magnetic disk storage media, optical storage media, flash-memory devices, and other non-transitory media.

In operation, the CPU 402 fetches, decodes, and executes instructions, and transfers information to and from other resources via the computer's main data-transfer path, system bus. Such a system bus connects the components in the computing system 400 and defines the medium for data exchange. Memory devices coupled to the system bus include random access memory (RAM) and read only memory (ROM). Such memories include circuitry that allows information to be stored and retrieved. The ROMs generally contain stored data that cannot be modified. Data stored in the RAM can be read or changed by CPU or other hardware devices. Access to the RAM and/or ROM may be controlled by memory controller. The memory controller may provide an address translation function that translates virtual addresses into physical addresses as instructions are executed.

In addition, the computing system can contain peripherals controller responsible for communicating instructions from the CPU to peripherals, such as, printer, keyboard, mouse, and data storage drive. Display, which is controlled by a display controller, is used to display visual output generated by the computing system. Such visual output may include text, graphics, animated graphics, and video. The display controller includes electronic components required to generate a video signal that is sent to display. Further, the computing system can contain network adaptor which may be used to connect the computing system to an external communications network.

As is well understood by those skilled in the art, the Internet is a worldwide network of computer networks. Today, the Internet is a public and self-sustaining network that is available to many millions of users. The Internet uses a set of communication protocols called TCP/IP (i.e., Transmission Control Protocol/Internet Protocol) to connect hosts. The Internet has a communications infrastructure known as the Internet backbone. Access to the Internet backbone is largely controlled by Internet Service Providers (ISPs) that resell access to corporations and individuals.

The Internet Protocol (IP) enables data to be sent from one device (e.g., a phone, a Personal Digital Assistant (PDA), a computer, etc.) to another device on a network. There are a variety of versions of IP today, including, e.g., IPv4, IPv6, etc. Other IPs are no doubt available and will continue to become available in the future, any of which can be used without departing from the scope of the invention. Each host device on the network has at least one IP address that is its own unique identifier and acts as a connectionless protocol. The connection between end points during a communication is not continuous. When a user sends or receives data or messages, the data or messages are divided into components known as packets. Every packet is treated as an independent unit of data and routed to its final destination—but not necessarily via the same path.

The Open System Interconnection (OSI) model was established to standardize transmission between points over the Internet or other networks. The OSI model separates the communications processes between two points in a network into seven stacked layers, with each layer adding its own set of functions. Each device handles a message so that there is a downward flow through each layer at a sending end point and an upward flow through the layers at a receiving end point. The programming and/or hardware that provides the seven layers of function is typically a combination of device operating systems, application software, TCP/IP and/or other transport and network protocols, and other software and hardware.

Wireless networks can incorporate a variety of types of mobile devices, such as, e.g., cellular and wireless telephones, PCs (personal computers), laptop computers, wearable computers, cordless phones, pagers, headsets, printers, PDAs, etc. For example, mobile devices may include digital systems to secure fast wireless transmissions of voice and/or data. Typical mobile devices include some or all of the following components: a transceiver (for example a transmitter and a receiver, including a single chip transceiver with an integrated transmitter, receiver and, if desired, other functions); an antenna; a processor; display; one or more audio transducers (for example, a speaker or a microphone as in devices for audio communications); electromagnetic data storage (such as ROM, RAM, digital data storage, etc., such as in devices where data processing is provided); memory; flash memory; and/or a full chip set or integrated circuit; interfaces (such as universal serial bus (USB), coder-decoder (CODEC), universal asynchronous receiver-transmitter (UART), phase-change memory (PCM), etc.). Other components can be provided without departing from the scope of the invention.

Wireless LANs (WLANs) in which a mobile user can connect to a local area network (LAN) through a wireless connection may be employed for wireless communications. Wireless communications can include communications that propagate via electromagnetic waves, such as light, infrared, radio, and microwave. There are a variety of WLAN standards that currently exist, such as Bluetooth®, IEEE 802.11, and the obsolete HomeRF.

By way of example, Bluetooth products may be used to provide links between mobile computers, mobile phones, portable handheld devices, personal digital assistants (PDAs), and other mobile devices and connectivity to the Internet. Bluetooth is a computing and telecommunications industry specification that details how mobile devices can easily interconnect with each other and with non-mobile devices using a short-range wireless connection. Bluetooth creates a digital wireless protocol to address end-user problems arising from the proliferation of various mobile devices that need to keep data synchronized and consistent from one device to another, thereby allowing equipment from different vendors to work seamlessly together.

An IEEE standard, IEEE 802.11, specifies technologies for wireless LANs and devices. Using 802.11, wireless networking may be accomplished with each single base station supporting several devices. In some examples, devices may come pre-equipped with wireless hardware or a user may install a separate piece of hardware, such as a card, that may include an antenna. By way of example, devices used in 802.11 typically include three notable elements, whether or not the device is an access point (AP), a mobile station (STA), a bridge, a personal computing memory card International Association (PCMCIA) card (or PC card) or another device: a radio transceiver; an antenna; and a MAC (Media Access Control) layer that controls packet flow between points in a network.

Computing system, described above, can be deployed as part of a computer network used to achieve the desired technical effect and transformation. In general, the above description for computing environments applies to both server computers and client computers deployed in a network environment. FIG. 3B illustrates an exemplary illustrative networked computing environment 400, with a server in communication with client computers via a communications network 450. As shown in FIG. 3B, server 410 may be interconnected via a communications network 450 (which may be either of, or a combination of a fixed-wire or wireless LAN, WAN, intranet, extranet, peer-to-peer network, virtual private network, the Internet, or other communications network) with a number of client computing environments such as tablet personal computer 402, mobile telephone, smart phone 404, diagnostic device 406, personal computer 402, and personal digital assistant 408. In a network environment in which the communications network 450 is the Internet, for example, server 410 can be dedicated computing environment servers operable to process and communicate data to and from client computing environments via any of a number of known protocols, such as, hypertext transfer protocol (HTTP), file transfer protocol (FTP), simple object access protocol (SOAP), or wireless application protocol (WAP). Other wireless protocols can be used without departing from the scope of the disclosure, including, for example Wireless Markup Language (WML), DoCoMo i-mode (used, for example, in Japan) and XHTML Basic. Additionally, networked computing environment 400 can utilize various data security protocols such as secured socket layer (SSL) or pretty good privacy (PGP). Each client computing environment can be equipped with operating system 438 operable to support one or more computing applications, such as a web browser (not shown), or other graphical user interface (not shown), or a mobile desktop environment (not shown) to gain access to server computing environment 400.

In operation, a user (not shown) may interact with a computing application running on a client computing environment to obtain desired data and/or computing applications. The data and/or computing applications may be stored on server computing environment 400 and communicated to cooperating users through client computing environments over exemplary communications network 450. The computing applications, described in more detail below, are used to achieve the desired technical effect and transformation set forth. A participating user may request access to specific data and applications housed in whole or in part on server computing environment 400. These data may be communicated between client computing environments and server computing environments for processing and storage. Server computing environment 400 may host computing applications, processes and applets for the generation, authentication, encryption, and communication data and applications and may cooperate with other server computing environments (not shown), third party service providers (not shown), network attached storage (NAS) and storage area networks (SAN) to realize application/data transactions.

### EXAMPLES

A user sets up a diagnostic device by selecting the device to be used as part of the system. The device is then associated with one or more of each of an audio and/or visual output. Next the user selects an output for the diagnostic device, such as a screen of the associated electronic device, a doctor's office, a healthcare provider, a relative, a health buddy, a caretaker, etc.

Thus, for example, during set-up, a user could identify the associated device as a blood pressure monitor device, an audio and/or visual output, for example, a musical selection made by the user and/or a video clip of a grandchild playing and the audio/video may or may not synchronized to the diagnostic measurement. Finally, the user can direct the result to be displayed on the associated electronic device for viewing by the user.

In another example, during set-up, a user could identify the associated device as a blood glucose monitor, an audio and/or visual output, for example, a musical selection made by the user and/or images from a favorite travel location. Finally, the user can direct the result to be transmitted to another location, such as the healthcare provider's office or a son or daughter that is monitoring the health status of a parent user.

In yet another example, during set-up, a user could identify a first associated diagnostic device as a blood glucose monitor, an audio and/or visual output, and a second associated diagnostic device, such as a heart rate monitor, which is associated with a second audio and/or visual output, which can be the same or different that the audio and/or visual output associated with the first diagnostic device. Additional diagnostic devices can be associated as desired. Finally, the user can direct the measured biological parameters from each associated diagnostic device be transmitted to one or more other locations, which are not necessarily overlapping.

During use, for example, a user could select an associated diagnostic device from which measurement is to be taken, or that process can occur automatically or semi-automatically as a result of proximity of the associated diagnostic device to a secondary device. Once the diagnostic device is detected, or the process is launched, an audio and/or visual output is presented (e.g., music plays, or images are displayed). Once the diagnostic device acquires the target diagnostic information, the acquired information is automatically transmitted to the one or more locations identified during set-up.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A system for acquiring medical information comprising:
a first biological parameter detection device configurable to acquire a biological parameter; and
an electronic device having an electronic device memory, at least one of a display for generating a visual output and a speaker for generating an audio output, and a communicator configurable to establish a communication link with the first biological parameter detection device, wherein the electronic device is configurable to deliver at least one of a first visual and a first audio output in response to a start command.

2. The electronic system of claim 1 wherein the at least one of the display and/or the speaker is pre-selected by a user.

3. The electronic system of claim 1 wherein the at least one of the display and/or the speaker output is automatically selected.

4. The electronic system of claim 1 wherein the at least one of the display and/or the speaker output is selected by a caretaker.

5. The electronic system of claim 1 wherein the first biological parameter detection device is selected from a group comprising: blood pressure monitor, glucose monitor, heart rate monitor, ECG monitor, respiratory monitor, blood oxygen monitor, body temperature monitor, and skin galvanic response monitor.

6. The electronic system of claim 1 wherein the first biological parameter detection device is configurable to provide the acquired biological parameter to a third party device.

7. The electronic system of claim 1 further comprising a second biological parameter detection device configurable to acquire a second biological parameter wherein the electronic device is configurable to generate at least one of a second visual output and a second audio output and further wherein the electronic device is configurable to deliver the second output in response to activation of the second biological parameter detection device.

8. The electronic system of claim 7 wherein the second biological parameter detection device is selected from a group comprising: blood pressure monitor, glucose monitor, heart rate monitor, ECG monitor, respiratory monitor, blood oxygen monitor, body temperature monitor, and skin galvanic response monitor.

9. The electronic system of claim 7 further wherein the second output is different than the first output.

10. The electronic system of claim 1 wherein a playback of the first visual output and/or the first audio output is synchronized to the biological measurement.

11. A method for acquiring a biological parameter comprising:
activating a first biological parameter detection device configured to acquire a first biological parameter;
activating an output of an electronic device having a device memory, at least one of a display for generating a visual output and a speaker for generating an audio output, and a communicator configurable to establish a communication link with the first biological parameter detection device;
measuring a biological parameter with the first biological parameter detection device;
determining an output associated with the first biological parameter detection device; and
delivering the output associated with the first biological parameter detection device on the electronic device concurrently with the step of measuring.

12. The method of claim 11 further comprising pre-selecting the output of the electronic device.

13. The method of claim 11 comprising selecting the first biological parameter detection device from the group comprising: blood pressure monitor, glucose monitor, heart rate monitor, ECG monitor, respiratory monitor, blood oxygen monitor, body temperature monitor, and skin galvanic response monitor.

14. The method of claim 11 further comprising:
activating a second biological parameter detection device configured to acquire a second biological parameter;
determining an output associated with the second biological parameter detection device; and
delivering an output associated with the second biological parameter detection device on the electronic device concurrently with the step of measuring.

15. The method of claim 14 comprising selecting the second biological parameter detection device from the group comprising: blood pressure monitor, glucose monitor, heart rate monitor, ECG monitor, respiratory monitor, blood oxygen monitor, body temperature monitor, and skin galvanic response monitor.

16. The method of claim 14 further wherein the output associated with the first biological parameter detection device is different than the output associated with the second biological parameter detection device.

17. The method of claim 11 comprising providing the acquired biological parameter to a third party.

18. A machine readable medium containing machine readable instructions that, when executed by a computing device, cause the computing device to perform a method, the method comprising:
activating a first biological parameter detection device configured to acquire a first biological parameter;
activating an output of an electronic device having a device memory, at least one of a speaker and a display, and a communicator configurable to establish a communication link with the first biological parameter detection device;
measuring a biological parameter with the first biological parameter detection device;
determining an output associated with the first biological parameter detection device; and
delivering the output associated with the first biological parameter detection device on the electronic device concurrently with the step of measuring.

19. The machine readable medium of claim 18 wherein the method further comprises pre-selecting an output.

20. The machine readable medium of claim 18 wherein the method further comprises selecting the first biological parameter detection device from a group comprising: blood pressure monitor, glucose monitor, heart rate monitor, ECG monitor, respiratory monitor, blood oxygen monitor, body temperature monitor, and skin galvanic response monitor.

21. The machine readable medium of claim 18 wherein the method further comprises:
activating a second biological parameter detection device configured to acquire a second biological parameter;
determining an output associated with the second biological parameter detection device; and
delivering an output associated with the second biological parameter detection device on the electronic device concurrently with the step of measuring.

22. The machine readable medium of claim 21 wherein the method further comprises selecting the second biological parameter detection device from a group comprising: blood pressure monitor, glucose monitor, heart rate monitor, ECG monitor, respiratory monitor, blood oxygen monitor, body temperature monitor, and skin galvanic response monitor.

23. The machine readable medium of claim 18 wherein the method further comprises providing the acquired biological parameter to a third party.
